(19) United States
(12) Patent Application Publication
(10) Pub. No.: US 2022/0136973 A1
(43) Pub. Date: May 5, 2022

(54) LINEAR POLYMER AFFINITY AGENT SENSOR FOR SURFACE-ENHANCED RAMAN SPECTROSCOPY AND METHOD USING THE SAME

(71) Applicant: Regents of the University of Minnesota, Minneapolis, MN (US)

(72) Inventors: Rebeca S. Rodriguez, St. Paul, MN (US); Victoria M. Szlag, St. Paul, MN (US); Theresa M. Reineke, Vadnais Heights, MN (US); Christy L. Haynes, Minneapolis, MN (US)

(21) Appl. No.: 17/514,016

(22) Filed: Oct. 29, 2021

Related U.S. Application Data
(60) Provisional application No. 63/107,117, filed on Oct. 29, 2020.

(51) Int. Cl. G01N 21/65; G01N 33/543

(52) U.S. Cl. CPC G01N 21/658; G01N 33/553; G01N 33/54373

(57) ABSTRACT

Methods and systems related to a linear polymer affinity agent sensor for SERS are disclosed. Use of the sensor may include mixing a linear polymer affinity agent in a sample solution, subjecting a metal substrate to the sample solution to attach the linear polymer affinity agent to the metal substrate, generating, via Raman Spectroscopy, spectral data representing the at least one linear polymer affinity agent attached to the metal substrate, and determining whether two or more analytes are present in the solution at respective minimum threshold concentrations based on the spectral data.

### CONTINUING APPLICATION DATA

This application claims the benefit of U.S. Provisional Application Ser. No. 63/107,117, filed Oct. 29, 2020, which is incorporated by reference herein.

### GOVERNMENT FUNDING

This invention was made with government support under DMR-1420013 awarded by the National Science Foundation and under OD017982 awarded by the National Institutes of Health. The government has certain rights in the invention.

### SUMMARY

Typical affinity agents used in analytical techniques are anchored onto a substrate and are not freely movable in the test solution containing the analyte. Such affinity agents are also usually deigned to be highly specific to the target analyte.

Applicant has found that by using linear polymers with suitable functional groups, the polymers may be allowed to freely move in the solution and to bind to the analyte by hydrogen bonding. A relatively high concentration of polymer may be present in the solution, allowing for the capture of a greater amount and/or larger number of analytes. The polymer-analyte complex may then be applied to a sensing substrate for spectral analysis, e.g., by surface-enhanced Raman scattering (SERS). The analytes may be any suitable small molecules that are capable of hydrogen bonding with the functional groups of the polymer. Two or more analytes may be sensed at the same time.

The system and method of the present disclosure involve the use of a polymer affinity agent, a sensing substrate, and SERS. The system and method of the present disclosure are suitable for detection of multiple analytes at the same time. The system and method of the present disclosure are suitable for detection of analyte molecules that are capable of hydrogen bonding. The system and method of the present disclosure are particularly suitable for detection of contaminants in foods and beverages.

Detection of mycotoxins, for example two or more mycotoxins simultaneously, is of particular interest. The number of known mycotoxins is very high, and various mycotoxins may be present in the same food or beverage product. However, due to their varying molecular structures, analysis of multiple mycotoxins at the same time is challenging, and often different mycotoxins need to be analyzed separately. For example, while various analytical techniques have been used to individually detect deoxynivalenol (DON) or ochratoxin A (OTA) at low limits of detection, direct multiplex detection of these toxins together is new. Collecting data in other types of multiplex sensing has often involved post-measurement chemometric analyses to distinguish between different toxins. Various aspects of the present disclosure relate to a linear polymer affinity agent used to directly detect both DON and OTA simultaneously via surface-enhanced Raman scattering without the need for chemometric analysis. Paired with density functional theory (DFT), the vibrational stretches of each small molecule are predicted, which provides additional insight on association of the analyte and polymers at the molecular level. This multiplex sensing scheme is simple, relatively inexpensive, and requires minimal analysis, displaying the overall potential a linear polymer affinity agent has for detecting various classes of small molecules.

In one aspect, the present disclosure relates to a method of using a sensor. The method includes mixing a linear polymer affinity agent in a sample solution. The method also includes subjecting a metal substrate to the sample solution to attach the linear polymer affinity agent to the metal substrate. The method may also include generating, via Raman Spectroscopy, spectral data representing the at least one linear polymer affinity agent attached to the metal substrate. The method also includes determining whether two or more analytes, such as toxins, are present in the solution at respective minimum threshold concentrations based on the spectral data.

In another aspect, the present disclosure relates to a sensor. The sensor includes a metal substrate including a plasmonic metal. The sensor also includes at least one linear polymer affinity agent. In one exemplary embodiment, the linear polymer affinity agent is synthesized via polymerization of N-(2-aminoethyl) methacrylamide hydrochloride (polymerization of AEMA, or pAEMA). The linear polymer affinity agent may bind to one or more analytes by hydrogen bonding. The linear polymer affinity agent may be attached to the metal substrate.

In another aspect, the present disclosure relates to a method of calibrating a sensor. The method includes subjecting a metal substrate to a calibrating solution including at least one linear polymer affinity agent and at least two analytes at respective known concentrations. The method also includes generating, via surface-enhanced Raman spectroscopy, spectral data representing the at least one linear polymer affinity agent being bound to the at least two toxins and being attached to the metal substrate. The method also includes generating calibration data based on the spectral data to detect the at least two analytes at respective minimum threshold concentrations. The calibration data includes identification of different peaks associated with each analyte.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIGS. 1A-B are illustrative images of multiplex detection on film over nanospheres (FON) surface-enhanced Raman scattering (SERS) substrates. FIG. 1A. Image of in-solution interactions occurring with a polymer affinity agent and two small molecule toxins. The complexed molecules attach onto the gold FON surface. FIG. 1B. Molecular structures for the methacrylamide polymer (pAEMA) and two small molecule toxins used in this study (deoxynivalenol and ochratoxin A).

FIG. 2A shows non-resonant computed Raman spectra for both DON and OTA. The modeled molecules are numbered. FIG. 2B is an enlarged image of DON molecular structure with labels corresponding to vibrational band assignments. FIG. 2C is an enlarged image of OTA molecular structure with labels corresponding to vibrational band assignments.

FIG. 3 shows experimental spectra of DON and pAEMA₂₉ complex at varying concentrations of DON. Each spectrum is an average of 15 spectra captured in the same conditions.

FIG. 4 shows experimental spectra of OTA and pAEMA₂₉ complex at varying concentrations of OTA. Each spectrum is an average of 15 spectra captured in the same conditions. The grey boxes highlight any spectral changes seen different from that of the control spectra.

FIG. 5A shows experimental spectra of both DON and OTA with pAEMA₂₉ complex at relevant regulatory concentrations 1 ppm and 0.005 ppm for DON and OTA respectively.

FIG. 5B shows the legend and schematic diagram for multiplex sensing experiment.

FIG. 6A shows enlarged multiplex spectra highlighting the OTA stand-alone peaks. FIG. 6B shows hypothesized interactions between polymer and OTA at 1535 cm⁻¹ shift based on vibrational animations in Gaussian. FIG. 6C shows hypothesized interactions between polymer and OTA at 916 cm⁻¹ shift based on vibrational animations in Gaussian.

FIG. 7A shows enlarged multiplex spectra highlighting the DON stand-alone peaks.

FIG. 7B shows hypothesized interactions between polymer and DON at 1450 cm⁻¹ shift and 1245 cm⁻¹ shift based on vibrational animations in Gaussian. FIG. 7C shows hypothesized interactions between polymer and DON at 1245 cm⁻¹ based on vibrational animations in Gaussian. FIGS. 7D. and E show hypothesized interactions between polymer and DON at 1142 cm⁻¹ and 1145 cm⁻¹ shift based on vibrational animations in Gaussian.

FIG. 8A shows raw heat flow injections of DON into pAEMA₂₉ and buffer solutions.

FIG. 8B shows raw heat flow injections of OTA into pAEMA₂₉ and buffer solutions. FIG. 8C is an ITC profile integrated and subtracted for both OTA and DON done in the same experimental run. A large heat at injection is seen for both toxins with pAEMA₂₉, indicating that both toxins kinetically bind to the polymer.

FIG. 9 shows a non-resonant Raman computed spectrum for the AEMA monomer at a 785 nm laser wavelength, using BP86 functional (Becke 1988 exchange functional and Perdew 86 correlation functional) model and TZP (triple zeta with 1 polarization function) basis set.

### DETAILED DESCRIPTION OF ILLUSTRATIVE EMBODIMENTS

In general, the present disclosure relates to multiplex surface-enhanced Raman scattering detection of one or more analytes using a linear polymer affinity agent and a sensing substrate. The present disclosure further relates to polymer affinity agents including one or more functional groups capable of hydrogen bonding, and to use of the affinity agents to detect analytes that are capable of hydrogen bonding with the functional groups of the polymer. The system and method of the present disclosure are suitable for detection of multiple analytes at the same time. The system and method of the present disclosure are particularly suitable for detection of contaminants in foods and beverages. Exemplary analytes include mycotoxins, cyanogenic glycosides, and other small molecule toxins.

As an example, the system and method of the present disclosure can be used to detect deoxynivalenol (DON) and ochratoxin A (OTA) with a linear polymer affinity agent. While various analytical techniques have been used to individually detect DON and OTA at low limits of detection, direct multiplex detection of these toxins together is new. Collecting data in other types of multiplex sensing has often involved post-measurement chemometric analyses to distinguish between different toxins. Various aspects of the present disclosure relate to a linear polymer affinity agent used to directly detect both DON and OTA simultaneously via surface-enhanced Raman scattering without the need for chemometric analysis. Paired with density functional theory (DFT), the vibrational stretches of each small molecule are predicted, which provides additional insight on association of the analyte and polymers at the molecular level. This multiplex sensing scheme is simple, relatively inexpensive, and requires minimal analysis, displaying the overall potential a linear polymer affinity agent has for detecting various classes of small molecules.

This disclosure describes the use of basic molecular hypotheses to design a linear polymer affinity agent that can bind multiple targets. According to an embodiment, the linear polymer affinity agent includes repeating monomer units, which include one or more functional groups capable of hydrogen bonding with an analyte. For example, the linear polymer affinity agent may include repeating monomer units that have an amine group, a hydroxyl group, or another functional group capable of hydrogen bonding. Further according to an embodiment, the linear polymer affinity agent has or may be reacted with a functional end group that is capable of bonding with the sensing substrate. For example, the linear polymer affinity agent may have or may be reacted with a functional end group that contains sulfur and is capable of bonding with the sensing substrate. Any suitable polymer backbone may be used. For example, the repeating monomer units may include (meth)acrylate, (meth)acrylamide, or any variation of acrylates and acrylamides. The size of the polymer may be selected such that the enhancement field of the SERS can be utilized. In some embodiments, the polymer has 10 or more, 15 or more, or 20 or more monomer repeating units. The polymer may have 40 or less, 35 or less, or 30 or less monomer repeating units. For example, the polymer may have from 10 to 35 or from 15 to 30 monomer repeating units.

One example of a suitable linear polymer affinity agent is pAEMA, which is a simple and inexpensive linear polymer. pAEMA may be synthesized to have from 10 to 35 or from 15 to 30 (e.g., 29) methacrylate units. pAEMA can be used to complex multiple analytes and anchor onto a sensing substrate through, for example, trithiocarbonate and gold interactions. For example, pAEMA can be used to complex at least two small molecule mycotoxins, DON and OTA, and anchor onto a sensing substrate through trithiocarbonate and gold interactions. This disclosure describes detection of both toxins individually using surface-enhanced Raman scattering (SERS), with no additional sensing probe molecule. Both toxins may be sensed simultaneously and visibly distinguished without any chemometric analysis of the data. The OTA Raman spectrum has been computationally modeled for the first time and added to the overall vibrational labeling of the DON Raman spectrum. Moreover, DFT can assist not only in the labeling of strong vibrational modes, but the ability to monitor the stretches in real-time facilitates clear conclusions about the fundamental interactions occurring between target and affinity agent during sensing. Hydrogen bonding may associate the two mycotoxins to the polymer through the amine groups. Additionally, hydrogen bonding may occur at multiple sites on the small molecules based on the location of the vibrational shifts. The polymer affinity agent may be selected such that multiple analytes (e.g., multiple toxins) are able to interact and complex to the polymer at concentrations relevant to their level of interest, such as possible regulation limits. For example, in the case of mycotoxins, both DON and OTA are able to complex to pAEMA at concentrations relevant to their regulation limits: 1 part per million (ppm) and 5 parts per billion (ppb) for DON and OTA, respectively. This further shows that linear polymer affinity agents can serve as unique capture agents due to their easily modifiable pendant groups, inexpensive nature, and non-specificity towards solely one target.

Various aspects of the present disclosure relate to polymers used as affinity agents to capture and detect a wide variety of analytes. Synthetic affinity agents are relatively inexpensive, robust, and are readily synthesized to tune and exploit certain interactions. Whether in conjunction with other affinity agents for increased specificity or synthesized specifically to bind to a target, these agents can be used to identify and detect biological toxins, food contaminants, and other small molecules.

Molecular imprinting polymers (immobilizing a target as a template in a polymer matrix) has long served as the synthetic mechanism to generate polymer-based affinity agents for sensors. However, these systems may be difficult to characterize and reproduce, due to their insoluble nature, and may be challenging to use as sensors in food samples with significant background signal due to the amount of other small molecules, proteins, sugars, etc. masking sensing identification, i.e. complex matrices. In addition, because they are cast to bind a specific target, these polymer templates cannot detect multiple targets at once without adding additional synthetic steps. These relatively thick polymer templates make it difficult to use surface analytical techniques for target detection, for example, due to the majority of the sensing volume being occupied by the polymer matrix rather than the matrix with captured analytes.

On the other hand, linear polymer affinity agents facilitate synthetic control of the chain length of the polymer and multivalent display of functional groups, where each monomer repeat unit serves as a potential binding site for the target analyte. In one example, a single-point-attachment polymer affinity agent with pendant saccharide moieties on the repeat unit structure was designed to specifically bind to a pocket of a protein used as a bioterror agent. Leveraging simple chemistry with an attractive analytical technique like SERS, one can monitor binding of the target to the polymer in both purified and complex matrices. Expanding on this concept, if one allows for similar bonding interactions, the choice of the polymer repeat unit can facilitate multiplex capture of an entire class of molecules.

In light of multiplex detection, SERS may be used as a signal transduction mechanism because of its low limit of detection, its ability to provide a “fingerprint” unique to the analyte of interest, and its compatibility with aqueous samples. When an analyte is immobilized near a plasmonic metal surface, one can observe an enhanced intensity of the analyte's signature “fingerprint” due to the vibrational modes of the analyte inelastically scattering light. These surfaces supporting localized plasmons are usually characterized by nanoscale roughness to generate a small-volume, but intense, electromagnetic (EM) field. This EM field extends only a few nanometers from the plasmonic surface. Both affinity agent and target analyte may be contained within the enhancement field to fully take advantage of SERS capabilities. Polymer chain length of the affinity agent may be a factor when using polymer affinity agents for SERS detection. With a long chain length (e.g., greater than 40), the analyte signature may not be seen. With a short chain length (e.g., fewer than 10), insufficient repeat unit binding sites for target-analyte interaction due to dense packing of the short polymer chains at the substrate surface may occur.

Various aspects of this disclosure relate to the use of SERS and linear polymers as affinity agents for multiplex analyte detection. In some embodiments, the optimization of linear polymer affinity agents may include the order of polymer and target attachment to the sensing substrate to reach relevant levels of detection. While traditional affinity agents are often anchored to the sensing substrate first, polymer affinity agents may exhibit heightened flexibility in solution, which may enable optimal polymer-target binding that is generally not achievable when pre-anchored to the substrate. This increases the amount of analyte that can readily associate with the polymer. The potential of linear polymer affinity agents may be demonstrated, for example, by multiplex detection of two different small molecule targets of interest, such as deoxynivalenol (DON) and ochratoxin A (OTA). These molecules are mycotoxins, small molecule toxins that are naturally produced from fungi that contaminate various types of crops and feedstocks. Mycotoxins are an interesting class of small molecules to detect, for example, due to their toxicity at very low exposure levels and ability to biomagnify in the environment. Both DON and OTA toxins are relevant targets for detection due to their prevalence in food, dangerous effects on livestock and humans, and their toxicity at very low concentrations. Multiplex detection of the two, simultaneously, is also important because they both contaminate grains and grain products.

Deoxynivalenol (DON), also known as vomitoxin, is a toxin naturally produced by Fusarium bacteria species (F. graminearum and F. culmorum). It is one of the most common mycotoxin contaminants in grains such as wheat and corn. When ingested, DON is an immunotoxin and can cause severe dehydration due to vomiting and diarrhea. Some jurisdictions regulate the DON in the range of 1 ppm for humans.

Ochratoxin A (OTA), is a toxin naturally produced by various Aspergillus and Penicillium bacteria species (A. ochraceus, P. verrucosum, A. carbonarius, and A. nigier). OTA tends to contaminate various grains, pork, and alcoholic beverages such as beer and wine. OTA is a nephrotoxin, a teratogen, a potential carcinogen, and has been linked to neurodegenerative diseases. Some jurisdictions regulate OTA at a much lower concentration compared to DON, such as 5 ppb, due to its extremely adverse effects.

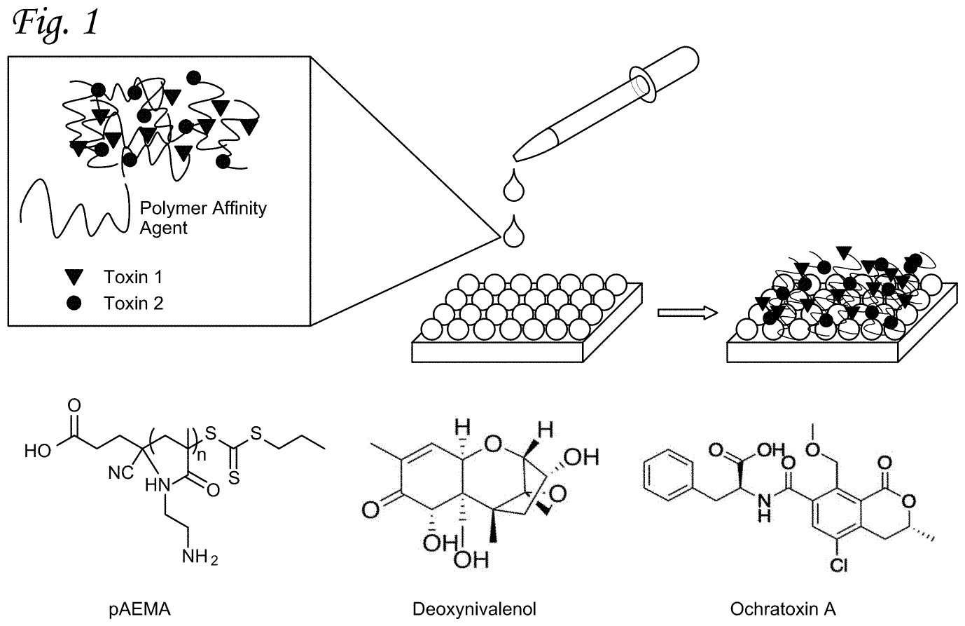

FIG. 1 shows a one example of a method for making a sensor including a metal substrate. The metal substrate may include a plasmonic metal. At least one linear polymer affinity agent may be synthesized via polymerization of N-(2-aminoethyl) methacrylamide hydrochloride (pAEMA) and attached to the metal substrate to bind to a toxin. In some embodiments, at least one polymer affinity agent includes pAEMA₂₉.

In some embodiments, the metal substrate may include at least one of gold, copper, aluminum, or silver. In a preferred embodiment, the metal substrate includes gold. The metal substrate may include one of a film of gold over a support substrate. The support substrate may include a silica nanosphere matrix, a colloidal gold substrate, or both.

According to an embodiment, the linear polymer affinity agent may be configured to bind to at least two analytes by hydrogen bonding. In some embodiments, at least one linear polymer affinity agent may be configured to bind to at least two mycotoxins. At least one linear polymer affinity agent may be configured to bind deoxynivalenol, ochratoxin A, or both.

The sensor may be used in any suitable manner. In one example, a method may include mixing a linear polymer affinity agent in a sample solution. The method may include hydrogen bonding of an analyte (e.g., two or more different analytes) to the linear polymer affinity agent. The method may also include subjecting a metal substrate to the sample solution to attach the linear polymer affinity agent to the metal substrate. The method may also include generating, via Raman Spectroscopy, spectral data representing the at least one linear polymer affinity agent attached to the metal substrate. The method may further include determining whether two or more toxins are present in the solution at respective minimum threshold concentrations based on the spectral data.

The linear polymer affinity agent may have at least 10 or at least 15 and up to 40 or up to 35 monomer repeating units. The linear polymer affinity agent may include one or more functional groups that are capable of hydrogen bonding. The linear polymer affinity agent may include methacrylamide. The linear polymer affinity agent may include amine groups, hydroxyl groups, or other functional groups capable of hydrogen bonding. In some embodiments, the linear polymer affinity agent may be synthesized via polymerization of N-(2-aminoethyl) methacrylamide hydrochloride.

In some embodiments, determining whether two or more analytes (e.g. toxins) are present may include determining whether two or more mycotoxins are present. Determining whether two or more analytes (e.g. toxins) are present may include determining whether deoxynivalenol, ochratoxin A, or both are present. Determining whether two or more analytes (e.g. toxins) are present may include detecting one or more hydrogen bonds between the linear polymer affinity agent and at least one of the analytes (e.g. toxins).

In some embodiments, determining whether two or more analytes (e.g. toxins) are present may include identifying a peak in the spectral data not associated with the linear polymer affinity agent or the two or more analytes (e.g. toxins). Determining whether two or more analytes (e.g. toxins) are present may include identifying bonds with a different functional group of each analyte (e.g. toxin) bonding with the linear polymer affinity agent.

Determining whether two or more toxins are present may include identifying a peak in a range from 300 to 1700 cm⁻¹ as corresponding to ochratoxin A. Determining whether two or more toxins are present may include identifying a peak in a range from 300 to 1800 cm⁻¹ as corresponding to deoxynivalenol.

The sensor may be calibrated using any suitable method. In one example, a method may include subjecting a metal substrate to a calibrating solution including at least one linear polymer affinity agent and at least two analytes at respective known concentrations. The method may also include generating, via Raman Spectroscopy, spectral data representing the at least one linear polymer affinity agent being bound to the at least two analytes and being attached to the metal substrate. The method may further include generating calibration data based on the spectral data to detect the at least two analytes at respective minimum threshold concentrations. The calibration data may include identification of different peaks associated with each analyte.

In some embodiments, at least two analytes may include two mycotoxins. At least two toxins may include deoxynivalenol, ochratoxin A, or both.

### Examples

A linear, methacrylamide polymer affinity agent was explored to capture two mycotoxins, deoxynivalenol (DON) and ochratoxin A (OTA), for multiplex surface-enhanced Raman scattering (SERS) detection. These mycotoxins are naturally occurring small molecules from fungi that can be dangerous at low concentrations. SERS detection was completed for each polymer-toxin complex at concentrations relevant to current safety regulation by the FDA: 1 ppm for DON and 5 ppb for OTA. Visibly distinguishable vibrational modes were observed in the multiplex spectra that were attributed to each mycotoxin individually, thus, not requiring any additional chemometric analysis. Density functional theory (DFT) was used to model DON and OTA to accurately label the vibrational modes in the experimental spectra as well as provide insight on the binding between both targets and the affinity agent. Fully modeled vibrations of these toxins are novel contributions due to OTA never being modeled and only a few published vibrational modes of DON. DFT guides empirical observations regarding hydrogen bonding at multiple sites of each mycotoxin target molecule through the amine groups on the polymer, confirming the capabilities of a single polymer affinity agent to facilitate multiplex detection of a class of molecules through less-specific interactions than traditional affinity agents.

### Materials.

N-(2-aminoethyl) methacrylamide hydrochloride (AEMA.HCl) was purchased from Polysciences, Inc, (Warrington, Pa.) and was purified by recrystallization in ethanol.³⁵ Deoxynivalenol from Fusarium graminearum and Fusarium culmorum cultures and ochratoxin A from Petromyces albertensis (OTA, ≥98%) were purchased from Sigma-Aldrich. The polymerization initiator, 4,4′-azobis(4-cyanovaleric acid) (V501, ≥98.0%), was purchased from Sigma-Aldrich as well. The chain transfer agent (CTA), 4-cyano-4-(propylsulfanylthiocarbonyl)-sulfanylpentanoic acid (CPP), was synthesized as previously reported in literature.³⁶ Silica nanospheres, with a 590 nm diameter (10% solids), were purchased from Bangs Laboratories, Inc (Fishers, Ind.). Pure gold (99.999%) was purchased from Kurt J. Lesker, (Clairton, Pa.). Purchased reagents did not undergo any further purification unless noted.

### Polymer Synthesis and Characterization.

Synthesis of this 29 repeat unit polymer (pAEMA₂₉) is fully described in a previous paper. (Szlag, V. M. et al. Isothermal Titration calorimetry for the Screening of Aflatoxin B1 Surface-Enhanced Raman Scattering Sensor Affinity Agents, Anal. Chem. 2018, 90 (22), 13409-13418.) Briefly, AEMA.HCl was dissolved in 90% 1 M acetate buffer, alongside 10% ethanol, the initiator, 4,4′-azobis(4-cyanovaleric acid) (V501), and the CTA 4-cyano-4-(propylsulfanylthiocarbonyl) sulfanylpentanoic acid (CPP). The reaction mixture was degassed via 3 cycles of freeze-pump-thaw and polymerized at 70-80° C. overnight (˜18 h). The polymerization was stopped by exposing the mixture to ambient air. The mixture was dialyzed in a 100-500 Da bag against 3 L of Milli-Q water for 24 h. This was then lyophilized resulting in a dry, light yellow solid with a yield of 78%. The polymer molecular weight was characterized via aqueous mobile phase (0.1 M Na₂SO₄ in 1.0 v % acidic acid) size exclusion chromatography (SEC). The instrument is an Agilent 1260 Infinity Quaternary LC System with Eprogen columns [CATSEC1000 (7 μm, 50×4.6), CATSEC100 (5 μm, 250×4.6), CATSEC300 (5 μm, 250×4.6), and CATSEC1000 (7 μm, 250×4.6)]. The system was equipped with a Wyatt HELEOS II light scattering detector (λ=662 nm) and an Optilab rEX refractometer (λ=658 nm).

### Isothermal Titration Calorimetry (ITC).

Isothermal titration calorimetry was carried out using an ITC-200 microcalorimeter. ITC measurements were performed using a MicroCal PEAQ-ITC Automated (Malvern Instruments, Westborough, Mass.) at 25° C. as previously discussed in previous literature. (Szlag, V. M. et al., Anal. Chem. 2018; Szlag, V. M. et al. Optimizing Linear Polymer Affinity Agent Properties for Surface-Enhanced Raman Scattering Detection of Aflatoxin B1, Mol. Syst. Des. Eng. 2019.)

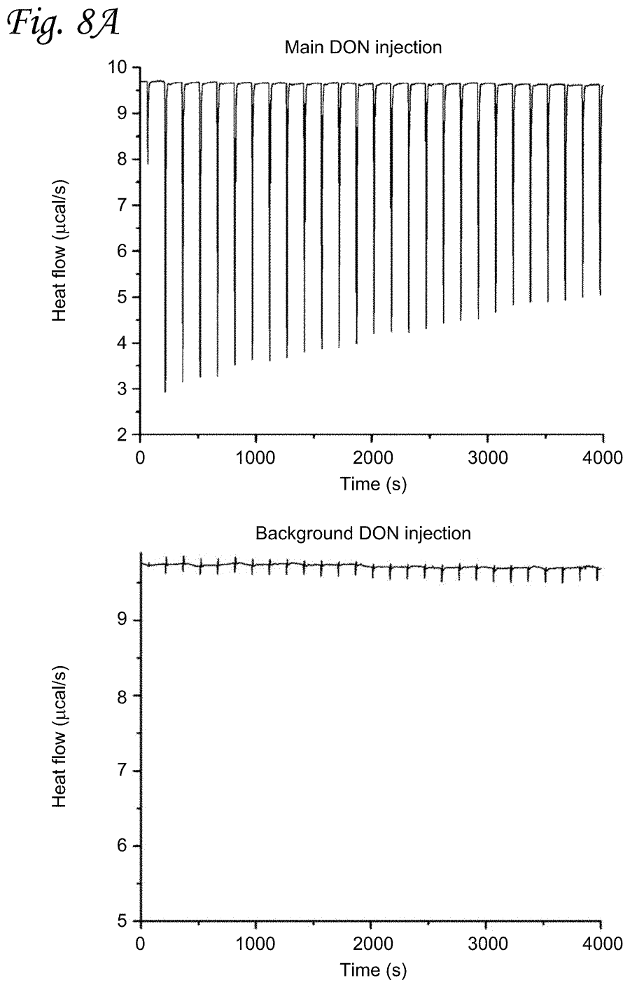

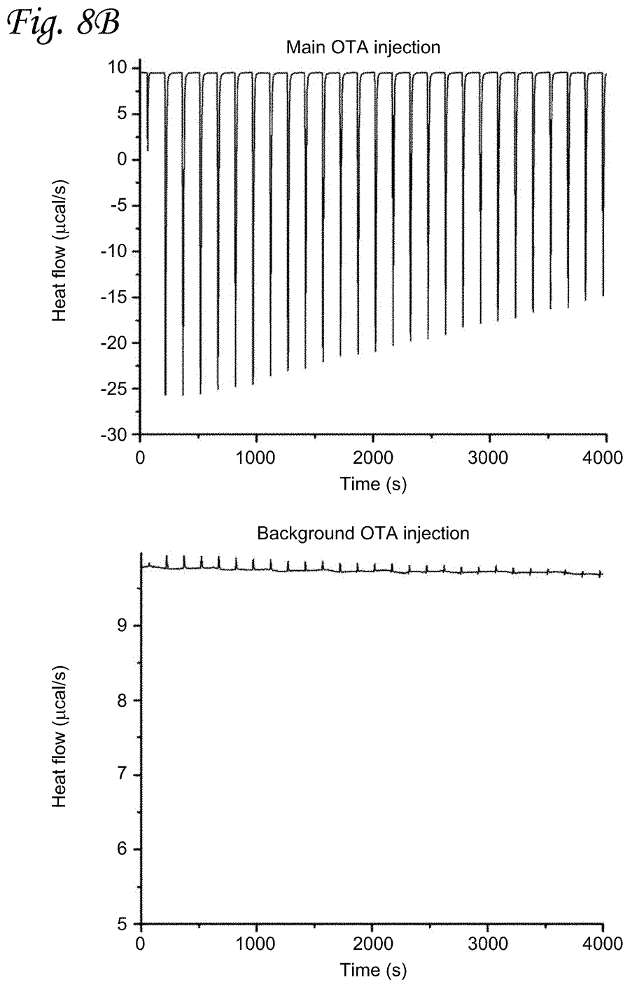

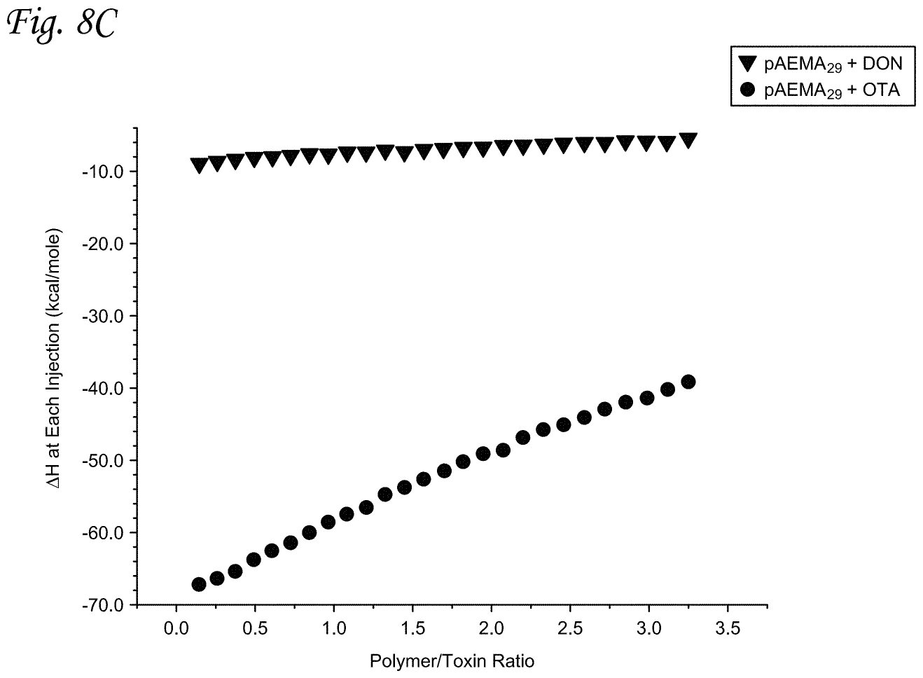

The sample cell and injection syringe were cleaned with 20% Contrad 70 detergent, water, and methanol. The instrument syringe was flushed with 10% bleach twice after each experiment. A 22 vol % dimethylsulfoxide, 16 vol % methanol, 62 vol % acetate buffer (pH 5) mixture was used to make a 4.0 mM polymer repeat unit solution and 0.26 mM OTA and DON samples. The instrument automatedly transferred polymer into the mycotoxin samples (or into blank solvent for the background titration). The titration used a 1.5 μL injection volume and 150 s injection intervals. Raw ITC profiles, as shown in FIGS. 8A and 8B, were measured as heat flow rate against time where each peak refers to the injected sample. Integration of these peaks, with respect to time, produced the final ITC plot depicting total heat absorption at each injection vs polymer repeat unit (RU)/toxin ratio as shown in FIG. 8C.

### Film Over Nanosphere (FON) Fabrication and Characterization.

FONs were fabricated as previously reported in literature. (Szlag, V. M. et al. Mol. Syst. Des. Eng. 2019; Kim, D. et al. Microfluidic-SERS Devices for One Shot Limit-of-Detection, Analyst 2014, 139 (13), 3227-3234; Le Ru, E. C. et al. Surface Enhanced Raman Scattering Enhancement Factors: A Comprehensive Study, J. Phys. Chem. C 2007, 111 (37), 13794-13803) 590-nm-diameter silica nanospheres were dropcast on 1 cm×1 cm silicon wafers to form a nanosphere mask. A 95.3 nm pure gold film was deposited under vacuum, measured by a quartz crystal microbalance (Denton Vacuum, Moorestown, N.J.). FONs with a localized surface plasmon resonance (LSPR) λₘₐₓ between 750 and 850 nm, measured using a fiber optic probe (Ocean Optics, Dunedin, Fla.) with a flat gold film as the reflective standard, were used.

### Surface-Enhanced Raman Scattering (SERS).

A 1 mM polymer solution (40:60 MeOH/water) was mixed with 50% by volume solutions of varying concentrations of DON and OTA and left to interact for 6 h. FON substrates were then incubated in 200 μL of the complexed mixture in a 24-wellplate for 18 h. Substrates were then washed with 1-2 mLs of Milli-Q water and air dried. Measurements were performed using a Snowy Range Instruments SnRI ORS System with a 785 nm laser, 9 mW incident power, and a 10 s integration time. Each condition was measured on three substrates, and five spots on each substrate was measured for a total of 15 averaged spectra. The FON average spectrum was baselined in OriginLab's Origin 9.1 (using eleven anchor points created by the first and second derivative with a Savitsky-Golay smoothing and connected by B-spline interpolation, with the same number of points as the input spectrum) and normalized by the incident power and integration time.

### Density Functional Theory.

Non-resonant Raman spectra of DON and OTA were calculated using density functional calculations in GaussView 6.0.16 with a basis set of B3LYP/6-311G++ (d, p) following previous literature that calculated a small number of DON vibrational bands. (Yuan, J. et al. Rapid Raman Detection of Deoxynivalenol in Agricultural Products, Food Chem 2017, 221, 797-802.) Rotational and vibrational bands were labeled for each molecule and can be found in Table 1.

TABLE 1
Vibrational band assignments for DON and OTA based on Gaussian calculations.

| Wave | |
|---|---|
| number | |
| shift (cm⁻¹) | Assignment |
| DON | |
| 201 | Rocking of C17—O5, OH bending on O5—H38 |
| 224 | Wagging of H31, H30, and H29 on C15, asymmetric stretching of H38 |
| 240 | Stretching of H38 and H36 |
| 243 | Wagging of H25 and H26 on C12 and of H33 and H34 on C17 |
| 248 | Bending of bond angle on O5 with H38 and O3 with H36 |
| 261 | Out of plane stretching of H38 on O5 and O3 with H36, and ring twist displacing H31 and |
| | H30 on C15 and C20 |
| 269 | Strong rocking of H38, ring breathing by C19, C21 twisting with H39, H40, and H41, |
| | wagging of H36 on O3 |
| 279 | Strong rocking of H29, H30, and H31 on C15 and rocking of H34 on C17 |
| 282 | C15 twist on H30, C20═C18 stretch, wagging of Hs on C21, C11 ring distortion |
| 293 | O1 twist, H26 and H25 twist in plane, asymmetric H23 and H24 stretching on C11 |
| 301 | Strong H29 and H30 twisting on C15, H37 rocking on O4, H40 rocking |
| 320 | Ring breathing C20, H39 twisting, C15 twisting, rocking of H29, H30, and H23 |
| 323 | Asymmetric bending of H29, H30, and H31 on C15, ring distortion C8 |
| 335 | Asymmetric bending of H34 and H33 on C17 and bond angle bending of H38 on O5, out |
| | of plane bending of H35 on C18, asymmetric bending of C18═C20, rocking of H25 |
| 361 | H35 rocking, H25 twist, asymmetric O1 stretch |
| 378 | Strong angle stretches of C16, asymmetric stretching of O6 and H37, H28 twist, in plane |
| | stretch of H34, H30, H23 |
| 389 | Strong angle stretches of C16, asymmetric stretching of O6 and H37, ring distortion C19, |
| | H twist on C21, O6═C19 wagging, asymmetric stretch of C9—C7, C15 wagging |
| 446 | O3—C13 asymmetric stretch, H twist of C11, ring breathing C18, C35 stretch, asymmetric |
| | O3 stretch |
| 471 | Ring breathing C20, O6═C19 stretch, H32 symmetric stretching to ring |
| 480 | Ring rocking C7, ring twist C9—C14, H23 and H24 wagging, symmetric angle bending |
| | C12—C8 and C10—C8, symmetric rocking of Hs on C15 |
| 494 | Symmetric ring breathing, H35 wagging, H34 wagging, H22 twisting, H38 bending |
| 521 | Strong wagging of H31 on O4, H41 on C21, H26 on C12, H33 on C17, asymmetric ring |
| | stretching C10 and C9 and C11 |
| 551 | Strong H37 wagging stretch on O4 |
| 561 | Asymmetric ring stretches C8, C10, H22, and strong wagging of H37 on O4 |
| 584 | Ring distortion/twist of C20, H32, H40 on C20, H24 on C11, and Hs on C12 |
| 622 | Ring breathing C7, H23 twist, H25 rocking, H40 rocking, H38 rocking, H40 rocking, H36 |
| | wagging |
| 626 | C8 ring distortion, H24 twist, H37 rocking, C20 twist, H32 twist |
| 665 | Ring breathing C18═C20, H35 stretch on C18, H33 on C17 in plane wagging, C16 ring |
| | breathing stretch, C35 wagging, in plane twist of Hs on C21 |
| 689 | Asymmetric ring distortion C14—C9, elongated stretch on C14 and H28, wagging of H38, |
| | asymmetric stretching of Hs on C17 |
| 747 | Hs wagging on C11, twisting of Hs on C15, symmetric angle bending of Hs on C10 and |
| | C12 |
| 774 | Ring distortion C13—C11, Hs on C11 twist, H36 on O3 bending, Hs on C14 angle bending |
| 803 | Ring twisting C9—C14, Hs on C21 twisting, H35 wagging on C18 |
| 853 | Ring breathing C10, Hs on C11 in plane stretch, H32 rocking, H35 bend |
| 861 | Asymmetric ring distortion C16 and C18, Rocking of H37 on O4, rocking of Hs on C21, |
| | H35 stretch on C18, asymmetric stretch of Hs on C15, asymmetric stretch of Hs on C11 |
| | and C13 |
| 874 | Asymmetric ring distortion of cyclopentane affecting H24 on C11 and Hs22 on C10, strong |
| | asymmetric stretching of H26 on C12 |
| 888 | Rocking of Hs on C15, asymmetric wagging of H35 on C18, rocking of H27 on H13 |
| 898 | Strong wagging of H35 on C18, strong wagging of H26 on C12, symmetric ring twisting, |
| | H32 twist, Hs on C21 twisting |
| 919 | Strong H34 twisting, slight ring distortion on C19—C20, strong H23 rocking, twisting of Hs |
| | on C12, twisting of Hs on C17, rocking of Hs on C15 |
| 925 | Hs on C11 twisting, Hs on C17 twisting, C9 ring breathing, Hs on C21 rocking, H27 on |
| | C13 wagging |
| 941 | Strong twisting of Hs on C12, symmetric stretch of H31 on C15, Hs rocking on C17, H22 |
| | on C10 wagging, slight ring distortion with C10 |
| 956 | Out of plane stretching of H35 on C18, Hs twisting on C21, wagging of Hs on C12, |
| | asymmetric stretching of C14, rocking of Hs on C15, ring stretch between C10, O2, and |
| | H14 bond angle |
| 966 | Out of plane stretching of H35 on C18, Hs twisting of C15, asymmetric ring stretching on |
| | C9, ring rocking inwards at C13, asymmetric ring stretching with C9 and C20, asymmetric |
| | stretch of H27 on C13 and C11, wagging of Hs on C21 |
| 980 | Strong wagging of H35 on C18, strong twisting of C22 on C10, asymmetric stretch of C25 |
| | on C12, ring rocking out of plane with C9 and C20, inward ring stretches with C10 and C9, |
| | C21 rocking. |
| 996 | Hs on C21 rocking out of plane, asymmetric stretching of Hs on C17, outward ring |
| | stretches with C18 and C16 |
| 1043 | Strong rocking of Hs on C15, C27 stretch on C13, asymmetric angle stretches of C17 and |
| | O5, H38 stretch of C5, H27 wag on C13, symmetric ring stretches of C9 and C20, ring |
| | stretch with C10, C14, and C7 |
| 1048 | Strong symmetric angle stretches of H27 and H24 on C13 and C11, H22 stretch of C10, |
| | slight ring stretching with O2 and C14, Hs rocking on C21, rocking of Hs on C15 |
| 1064 | Hs twisting on C11, H35 stretch on C18, Hs rocking on C21, rocking of H30 on C15, ring |
| | rocking with C16 and C18 |
| 1067 | Strong rocking of H23 on C11, rocking of Hs on C1, symmetric ring distortion with C11 |
| | and C13, rocking of Hs on C21 |
| 1068 | Strong asymmetric stretching of Hs on C21, asymmetric stretching of H35 on C18 and C20 |
| 1087 | Strong wagging of H36 on O3, rocking of Hs and O5 on C17, asymmetric stretching of |
| | ring with C10 and C14 on O2, twisting of Hs on C12, asymmetric stretching of O5 and |
| | C17 with H34 stretching in plane with C17 |
| 1090 | Wagging of H36 on O3, symmetric stretching of O5 and H33 on C17, rocking of Hs on |
| | C21, symmetric stretching of ring with C10 and C13, rocking of H32 on C16, stretching of |
| | C17 on C9, rocking of H35 on C18 |
| 1114 | Rocking of H28 on C14, twisting of Hs on C12, rocking of Hs on C15, ring stretching with |
| | C18, C14 and C9, rocking of Hs on C15 |
| 1119 | Strong twisting of Hs on C12 |
| 1142 | Symmetric stretching of O3 and C11 with H35 on O3 and H14 on C11, wagging of H38 on |
| | O5, asymmetric stretching of Hs on C21, asymmetric stretching of Hs on C12, rocking of |
| | Hs on C11, ring twisting with C7, C9, C16, and C13 |
| 1146 | Symmetric wagging of Hs on C12, symmetric bending of Hs on C15, wagging of H38 on |
| | O5, ring breathing with C11 and C7, asymmetric ring stretching with C16 and C14 on C9 |
| 1154 | Ring distortion with C16 displacing H32, twisting of Hs on C11 and twisting of C13, |
| | wagging of H38 on O5, asymmetric ring distortion with C7 and C10 |
| 1164 | Symmetric wagging of Hs on C15, in plane rocking of Hs on C12, rocking of H35 on C18, |
| | ring rocking of C7 and C14 |
| 1178 | Symmetric wagging of Hs on C12, rocking of Hs on C15, wagging of H38 on O5, wagging |
| | of H23 on C11, asymmetric ring stretches with C7, C10, and C9, ring breathing of pentane |
| | ring, rocking of H36 on O3 |
| 1195 | Strong twisting of Hs on C17, symmetric ring breathing on cyclohexene ring with wagging |
| | of H35 on C18, wagging of H38 on O5, stretching of H30 on C7, asymmetric stretching of |
| | C8 and C11 bonded to C7, wagging of H37 on O4 |
| 1216 | Strong wagging of H23 on C11 and H36 on O3, angle bending of H22 and H27 on |
| | C10—C13, symmetric stretching of Hs on 12 and C8 |
| 1233 | Strong wagging of H22 on C10, H36 on O3, stretch of H27 on C13, similar to previous |
| | mode |
| 1245 | Strong stretching of H22 on C10, symmetric stretching of H27 on C13 and O3 on C13, |
| | strong angle wagging of H38 on O5 and H33 on C17 |
| 1251 | Strong rocking of H32 on C16, symmetric stretching of H38 and H33 on O5—C17 |
| | respectively, strong stretching of H35 on C18, ring distortion of cyclohexene with |
| | asymmetric stretching of C19—C20 |
| 1264 | Strong rocking of H32 on C16, wagging of H38 on O5 and H33 on C17, twisting of Hs on |
| | C11, slight ring distortion on cyclohexane with C7—C9 |
| 1270 | Wagging of H24 on C11, wagging of H38 on O5, rocking of H35 on C18, slight ring |
| | distortion caused by C18—H35 and H32 on C16 |
| 1283 | Strong wagging of H32 on C16, strong H32 rocking on C16, wagging of H35 on C18, |
| | wagging of H24 on C11, ring distortion of cyclohexene caused by C16 and C18 |
| 1308 | Symmetric rocking of Hs on C11, twisting of Hs on C17, stretching of H22 on C10, |
| | wagging of H27 on C13, rocking of H28 on C14, ring distortion of cyclopentane |
| 1320 | Asymmetric twisting of Hs on C17, wagging of H22 and H32 |
| 1344 | Strong symmetric “windshield wiper” stretching of H24 and H27 on C11 and C13 |
| | respectively, ring distortion of cyclopentane due to C11 and C13 movement, wagging of |
| | H36 on O3 |
| 1348 | Strong rocking of H28, wagging of H35, “windshield wiper” motion of H22 and H27, |
| | asymmetric stretching of Hs on C17 |
| 1355 | Strong rocking of H22 on C10 and symmetric stretching of H27 on C13, not as strong, |
| | wagging of H28 on C14, slight ring twist on cyclopentane due to C10 movement |
| 1361 | Strong wagging of H32 on C16, wagging of H35 on C18, asymmetric Hs twisting on C17, |
| | rocking of C9 |
| 1384 | Rocking of H37 on O4, symmetric angle bending of C18—C14 displacing H35 and H28, |
| | rocking of Hs on C21, ring rocking of cyclohexene |
| 1399 | “windshield wiper” motion of Hs on C18—C14 (H35 and H28 respectively), wagging of |
| | H32, ring distortion of cyclohexene, wagging of H37 on O4 |
| 1413 | Symmetric angle breathing of all Hs on C21 “claw”, slight twisting of C14 |
| 1416 | Symmetric angle breathing of Hs on C15 “claw”, slight rocking of Hs on C12, slight |
| | rocking of C8 and H2 bonded to C10, and wagging of H27 on C13 |
| 1426 | Angle bending of H37 on O4 and C16 on O4, wagging of H27 on C13, wagging of H28, |
| | wagging of H37 on O4, out of plane symmetric angle breathing of Hs on C21, cyclohexene |
| | ring distortion, wagging of H32 on C16 |
| 1427 | Strong wagging of H27 on C13 and H36 on O3, wagging stretch of H37 on O4, slight |
| | symmetric angle breathing of Hs on C21, cyclopentane ring distortion with C13 |
| 1437 | Asymmetric stretching of Hs on C12 and C8—C12 bond, in plane angle bending “claw” Hs |
| | on C15, asymmetric stretching of H22 on C10 and C10—C8 bond, ring distortion of pentane |
| | ring with C8, slight wagging of Hs on C17 |
| 1450 | Asymmetric out of plane rocking of Hs on C17 and wagging of H38 on O5, wagging of |
| | H37 on O4, slight wagging of Hs on C11 |
| 1473 | Asymmetric twisting and wagging of Hs on C12 |
| 1488 | Asymmetric stretching and wagging of Hs on C21, slight wagging of Hs on C11 |
| 1490 | Scissoring of Hs on C11, asymmetric wagging of Hs on C21, asymmetric stretching of Hs |
| | on C15, asymmetric stretching of Hs on C17 |
| 1503 | Asymmetric wagging of Hs on C15, slight scissoring of Hs on C12, scissoring of Hs on |
| | C17, symmetric stretching of Hs on C11 |
| 1516 | Strong asymmetric wagging of H33 and H34 on C17, asymmetric twisting and stretching |
| | of Hs on C15, slight scissoring of Hs on C12 |
| 1517 | Strong twisting and stretching of Hs on C15, slight stretching of Hs on C12 |
| 1530 | Scissoring of Hs on C12, twisting of Hs on C15 |
| 1715 | Strong stretching of C18═C20 bond, ring distortion of cyclohexene due to double bond |
| | stretch, Hs wagging on C21, wagging of H28 on C14 |
| 1727 | Strong stretching of O6═C19 bond, opposite stretching of C20═C18 bond, wagging of H37 |
| | on O4, symmetric wagging of H35 on C18 and H41 on C21, ring distortion caused by |
| | strong C19═O6 bond |
| OTA | |
| 219 | Stretching of C28 and C22 in benzene ring and corresponding Hs, asymmetric stretching of |
| | C17—N7, displaced stretching of C23═O7 and O6═H46, twisting of Hs on C20, inward |
| | stretch of benzene ring with C11, and rocking of Hs on C18 |
| 221 | Strong twisting of Hs on C18, rocking of C9 displacing Hs |
| 240 | Symmetric rocking of C9 and C18 displacing their Hs, asymmetric stretching of benzene |
| | ring with C28 and C22, rocking of Hs on C20, rocking of Hs on C17, windshield wiper |
| | motion of C21═O5 and C17—H32, rocking of Hs on C18, and twisting of Hs on C9 |
| 265 | Twisting of Hs on C9, rocking of Hs on C18, rocking of O5═C21, asymmetric stretching of |
| | benzene ring with C11 and C14 |
| 293 | Strong angle breathing of C9—C10—C18, rocking of Hs on C18, outward stretch of benzene |
| | ring with C12 and C11, rocking of H40 on O3, out of plane rocking of H39 on N8 |
| 303 | Out of plane rocking of benzene ring with C19 and C12, strong angle breathing of |
| | O5═C21═N8—C17—C23, rocking of Hs on C17 and O6, symmetric stretch of Hs on C18 |
| 316 | Rocking of Hs on C18, asymmetric stretching of bonded rings: large stretch with C10 and |
| | out of plane rocking of benzene ring, stretching of C11, twisting of benzene ring with H43 |
| | and H44 |
| 336 | Strong wagging of O3—H40, wagging of O4 and H36, rocking of Hs on C20 |
| 357 | Strong wagging of Hs on C20, twisting of benzene ring with H41, H43, H45, and H44 |
| 366 | Rocking of Hs on C18, strong outward breathing stretch of benzene ring with C11 and C13, |
| | bond angle symmetric stretch of O3, C13, C12, and C16, strong rocking of H40 from O3, |
| | strong asymmetric stretch in bonded ring structures with O4 and C11 |
| 376 | Strong wagging of Hs on C20, angle breathing of N8, C17, and C20, wagging of H39, |
| | asymmetric stretching of H39 and H37, rocking of O7, asymmetric out of plane benzene |
| | stretch of C28 and C22 displacing their Hs, wagging of C21═O5 |
| 401 | Strong wagging of H39 on N8, wagging of Hs on C18, asymmetric symmetric of ring with |
| | C12 and C10, stretching of O3, rocking of Hs on C9, wagging of O4═C16, symmetric |
| | stretch of benzene ring with C11 and C13 |
| 415 | Asymmetric stretching of C25═C27 and C24═C26 displacing their Hs respectively |
| 426 | Strong wagging of H39 on N8, benzene ring breathing with C19 and C13, twisting of Hs |
| | on C9, ring breathing with C16 and C11 |
| 439 | Strong wagging of H39 on N8, slight twist of Hs on C9 |
| 458 | Strong twisting of Hs on C9, rocking of Hs on C18, stretching of H31 |
| 481 | Symmetric ring stretchings with O2 and H36 pulling, benzene ring breathing rocking with |
| | C19—H36 and C11, rocking of H40, rocking of C15 in benzene ring, symmetric ring |
| | stretch of H30—C9, and C15 |
| 492 | Inverted benzene ring stretching, asymmetric out of plane stretching of H43 and H45, |
| | symmetric stretching of H41 and H44, out of plan stretching of C22, rocking of H45, |
| | rocking of Hs on C20, rocking of H39, slight rocking of H30 |
| 512 | Out of plane rocking of H36, out of plane rocking of H46, symmetric twisting of bonded |
| | rings, rocking of Hs on C18, twisting of O4 and O3—H40, ring breathing of C24 |
| 520 | Strong twisting of H40 bonded to O3, asymmetric twisting of bonded rings, rocking of |
| | H36, rocking of H39, rocking of Hs on C18 |
| 557 | Angle breath between O6—H45, C23, and O7, ring distortion through C28 and C22, rocking |
| | of H39, rocking of H36 |
| 583 | Strong twist of Hs on C20, strong rocking of H46, rocking of H39, ring distortion through |
| | C22 and C28 |
| 592 | Strong rocking of H46 on O6 |
| 610 | Rocking of H's bonded to C9, twisting of ring due to C9 rocking, rocking of O5, scissoring |
| | of H46 and H39, rocking of H's bonded to C18, O4 stretch, slight ring breath at C28 and |
| | C22, twisting of ring at C13 and C11 |
| 621 | Rocking of H's bonded to C18, twisting of ring at C10, wagging of O4, ring distortion at |
| | C11, scissoring of H36 and H29, rocking of H39, slight rocking of H's bonded to C20, |
| | rocking of C9 |
| 635 | Strong inward ring breath at C27, C24, C25, C26 and displacement of bonded H's, slight |
| | rocking of H's bonded to C20 |
| 648 | Wagging of H46, rocking of H's bonded to C9 and C11 causing a slight ring distortion on |
| | both rings, rocking of H's bonded to C18, rocking of C16═O4 and O3—H40, slight ring |
| | breath at C25 and C26, wagging of H46 |
| 654 | Outward bond angle stretch of C23═O7 and O6 and H46, twisting of H's on C20, ring |
| | distortion at C11 and twisting of H29, ring twist displacing H43 |
| 697 | Ring breathing of both bonded rings, slight C11—C15 stretch, rocking of H's bonded to C18, |
| | wagging of H39, downward stretch of C21, rocking of H46 following ring breathing |
| | pattern |
| 714 | Asymmetric out of plane breathing of benzene ring, strong displacement of H41, H42, and |
| | H45 |
| 735 | Strong stretching of C23 and H46 and ring breathing at C26 and C27 |
| 739 | Out of plane rocking of C23 and H46 on O6, ring distortion at C14, rocking of H40 on O3 |
| 748 | Strong ring distortion at C14, slight out of plane rocking of hydrogens on benzyl ring, ring |
| | breathing at C15 and C13 with stretching of H40 on O3 |
| 767 | Strong hydrogen breathing of the benzyl ring at C26, rocking of C20 and C23 along with |
| | the hydrogens bonded |
| 787 | Strong wagging of H40 on O3 |
| 803 | Strong wagging of H40 on O3 and symmetric stretching of C14, C21, and C19 with a |
| | strong stretch of H36 |
| 812 | Strong out of plane symmetric stretching of C26 and H40, rocking of C13 and C12 bond |
| 822 | Ring Breathing at C26—C24 and C25—C27, symmetric stretching of C20 and C22 displacing |
| | the hydrogens on C20, symmetric stretching of C17—C23 bond and wagging of H46 |
| 833 | Symmetric inward stretches of rings at C19 and O2, wagging of H35, weak benzyl ring |
| | breathing at C27 and C22 |
| 857 | Strong out of plane asymmetric stretching of hydrogens (H41, H42, H43, H44, H45) in |
| | benzyl ring |
| 873 | Strong twisting of Hs on C20, C17—C20 bond stretching with H32 rocking, out of plane |
| | asymmetric stretching of hydrogens (H41, H42, H43, H44, H45), rocking of N8 and |
| | hydrogen bonded to it, out of plane rocking of H36, |
| 898 | In plane wagging of H's on C18, angle breath of C10 and C18, C9 stretching |
| 917 | Strong twisting of hydrogens on C20, strong rocking of H36, rocking of C17 and H32, |
| | bond angle stretch of C18—C10, rocking of H's on C18, rocking of H's on C9, slight ring |
| | breathing at C22 and C26 |
| 923 | Strong angle stretch of C18—C10, rocking of H's on C18, C10, and C9, rocking of H32, |
| | H42, H41, H45, slight stretching of N8═C21 |
| 927 | Strong rocking of H36, strong rocking of H29, twisting of H's on C18, slight ring distortion |
| | at C19 and C15, symmetric stretching of H42 and H45, asymmetrical stretching of H41 |
| 930 | Strong rocking of H36, asymmetric out of plane stretching of H's on benzyl ring with C24, |
| | twisting of H's on C18, out of plane rocking of C9 and H's bonded |
| 939 | Strong out of plane rocking of H36, asymmetric out of plane rocking of H's on benzyl ring |
| | with C24, outward stretching of H's bonded to C18 |
| 982 | Strong twisting of H's on C18, bond angle stretching at O2═C16═O4, ring distortion caused |
| | by O2, ring distortion at C19 and C15, rocking of O3—H40 |
| 985 | Strong out of plane rocking of H41 and H44 symmetrically and H42, H43, and H45 strong |
| | out of plane rocking asymmetric to the previous |
| 1005 | Asymmetric out of plane stretching of hydrogens on C25 benzene ring |
| 1017 | Strong ring breathing distortion at C28, C25, C24 |
| 1038 | Strong twisting of H's on C20, wagging of C17, slight asymmetric stretching of C25 |
| | benzene ring, C10 ring distortion |
| 1050 | Strong ring breathing with twist at C27 and C26 rocking all the H's bonded to the ring |
| 1055 | Strong rocking of H's on C20, ring wagging at H44, H45, H41, and subsequently H37 and |
| | C20, stretching of N8═C21═O5 and H39, rocking of H36, ring distortion caused by C9 |
| | rocking bonded to C11, C10—C18 stretch, inward symmetric stretch of H's on C18 |
| 1082 | Strong C9 H's stretch causing a strong inner ring distortion on C9, C10, C11, wagging of |
| | H39, asymmetric stretch of benzene ring at C11, wagging of H40 |
| 1105 | Wagging of H44, H42, H45, H43, H41 on benzyl ring, rocking of H's on C20, stretching of |
| | C17—C20 bond |
| 1120 | Symmetric wagging of H42, C22, H41 and H44, H45, H43, out of plane rocking of C20 |
| | and H's bonded, bond stretch of C17—N8, wagging of H30 |
| 1129 | Strong symmetric wagging of H33 and H38, bond stretch of C18—C10, wagging of H's |
| | bonded to C20 |
| 1151 | Strong bond stretch of C10—C9, rocking of H's on 18, inward stretch of O2═C16 bond, |
| | wagging of H39, N8═C21 bond stretch, wagging of H37 and H39, rocking of C17, |
| | wagging of H46 |
| 1170 | Strong symmetric wagging of H39 and H46, outward bond stretch of C10—C18, rocking of |
| | H's bonded to C18 and C10, slight stretching of H44 and H43 |
| 1183 | Strong inward stretching of H45 and H44, inward stretching of H43 and H41, wagging of |
| | H42 |
| 1185 | Wagging of H45, bond stretch between O6—C23, rocking of H's bonded to C18, rocking of |
| | H31 and H29, bond stretch of C10—C9, wagging of H40 |
| 1204 | Strong scissoring of H43 and H41, strong scissoring of H44 and H42 |
| 1211 | Strong rocking of H36, ring distortion caused by C15═C19 bond, wagging of H29 and |
| | H31, rocking of H's bonded to C18, asymmetric wagging of H's on C20, rocking of H's on |
| | C17, wagging of H43 and scissoring of H44 and H45, wagging of H46 |
| 1215 | Wagging of H36 and rocking of the Hs on C20, outward ring breathing at C15 and benzyl |
| | ring, wagging of H29, asymmetric bond stretching of C10—C9—C11, rocking of H40, |
| | scissoring of H41 and H43, inward wag of H44 and H42 |
| 1222 | Strong bond stretch of C22—C20 causing a ring distortion at C22, C27, C25, all hydrogens |
| | in benzyl ring are wagging inward, displacement of Hs bonded to C20 due to bond stretch, |
| | slight H39 wag |
| 1233 | Bond stretch of C22—C20 causing H's on C20 to asymmetrically wag, wagging of H32, |
| | outwards wagging of Hs on benzyl ring, and rocking of H31, twisting of H's on C9, |
| | symmetric breathing of ring caused by C14 and C11, C13═C12 bond stretch, Cc19═C15 |
| | bond stretch, wagging of H40, bond stretch of C12—C16 |
| 1243 | Strong twisting of H's on C9, wagging of H36, inward wagging of H31, scissoring of H's |
| | bonded to C18, wagging of H40 |
| 1288 | Twisting of H's bonded to C20, scissoring of H32 and H39, asymmetric bond stretching of |
| | N8═C21—C14, rocking of H36 causing a slight ring distortion at C19, wagging of H40, |
| | slight outward twisting of H42 and H44 on ring |
| 1302 | Strong wagging of H36, symmetric inward stretching of H's bonded to C9 and C10, slight |
| | rocking of H's bonded to C18, bond stretching in benzyl ring at C11═C15, C15═C19, |
| | C19═C14, symmetric wagging of H32 and H38 |
| 1303 | Strong wagging of H32 and H38, inward wagging of H's bonded to C9 and C10 |
| 1330 | Scissoring of H30 and H31, bond stretch of C13═O3, bond stretch of C11═C15═C19 all |
| | causing a ring distortion, wagging of H36, asymmetric bond stretching of C26—C12—C13 |
| | causing ring distortion, displacement of O3, wagging of H32, wagging of H's bonded to |
| | C20, slight ring distortion wagging H41, H43, H45, H44 and H42 |
| 1333 | inward bond angle stretch of H38 and C22, asymmetric bond stretching of C22—C24 and |
| | C29═C27 causing all the H's bonded in the ring too wag, rocking of H32, wagging of H46, |
| | wagging of H31 |
| 1350 | Strong wagging of H40 caused by bond stretch of O3═C13, strong wagging of H31, strong |
| | rocking of H's bonded to C9bond stretch of C12═C13 and C14═C19 causing a ring |
| | distortion and wagging of Hs on ring, twisting of H's on C18, slight wagging of H32 and |
| | H27 |
| 1352 | Twisting of H's on benzyl ring (strongest twist at H41), rocking of H32 |
| 1363 | Strong inward bond angle stretch of H46—O6—C23, scissoring of H's bonded to C17, |
| | rocking of H's bonded to C20, rocking of H39, slight twisting of H's on benzyl ring listed |
| | previously |
| 1372 | Wagging of H40, wagging of H29 and H31, inward ring breathing at C11═C12, wagging |
| | of H32, rocking of H's bonded to C20, slight twisting of Hs on benzyl ring listed previously |
| 1374 | Twisting of H's on benzyl ring (strongest twist at H44), rocking of H32, strong rocking of |
| | H's bonded to C20, wagging of H46, rocking of H40 |
| 1378 | Strong H32 wagging on C17, asymmetric wagging of Hs on C20, slight twisting of |
| | benzene ring Hs (H42, H44, etc.) |
| 1393 | Strong wagging of H31, wagging of H29, scissoring of H's bonded to C18 |
| 1407 | Wagging of H31, rocking of H's bonded to C9, inward bond stretch of H's bonded to C18, |
| | slight wagging of H30 and H36 |
| 1420 | Strong scissoring of H's bonded to C18, wagging of H31 |
| 1450 | Wagging of H40 and H36, bond angle stretching of C13—O3 and C15═C19 |
| 1468 | Scissoring of H30 and H20, slight scissoring of H33 and H35 |
| 1482 | Bond stretching of C24═C26 causing H's bonded to scissor, twisting of H's on same benzyl |
| | ring, scissoring of H's bonded to C20 |
| 1486 | Strong twisting of H's bonded to C18 |
| 1489 | Twisting of H's bonded C18, scissoring of H's bonded to C9, wagging of H40, C11—C9 |
| | bond stretch causing ring distortion and ring twisting at C11 and C14, wagging of H36, |
| | wagging of H39, scissoring of H's bonded to C20 |
| 1496 | Strong scissoring of H's bonded to C20, slight ring twisting wagging H45 and H44 |
| 1499 | Twisting of H's bonded to C18 |
| 1517 | Wagging of H39, outward stretching of H's bonded to C20 |
| 1527 | Strong symmetric stretching of benzene ring through H43 and H44 |
| 1606 | Strong symmetric bond stretch of C19═C14 and C11═C12 causing a ring distortion, |
| | wagging of H36, wagging of C29, slight bond stretch of O4═C16 |
| 1624 | Asymmetric bond stretching between C24═C22═C25 and C36═C28═C27, twisting of H's |
| | bonded to ring, rocking of H's bonded to C20 |
| 1635 | Asymmetric bond stretch of C14═C13═C12 and C19═C15═C11, wagging of H36 and H40, |
| | slight rocking of C9 |
| 1644 | Symmetric bond stretching of C36═C24 and C25═C27, causing a ring distortion and |
| | scissoring of bonded H's |
| 1722 | Strong bond stretch of O4═C16 causing outward ring distortion, bond stretch of C12═C11 |
| | and O3═C13, wagging of H40, bond stretch of O5═C21, slight wagging of H39 |
| 1747 | Strong bond stretch of O5═C21, wagging of H39, wagging of H40, bond stretching of |
| | O4═C16 distorting ring |
| 1814 | Strong bond stretch of C23═O7, scissoring of H46 and H32 |

### Example 1

### DFT Modeling of Deoxynivalenol and Ochratoxin A.

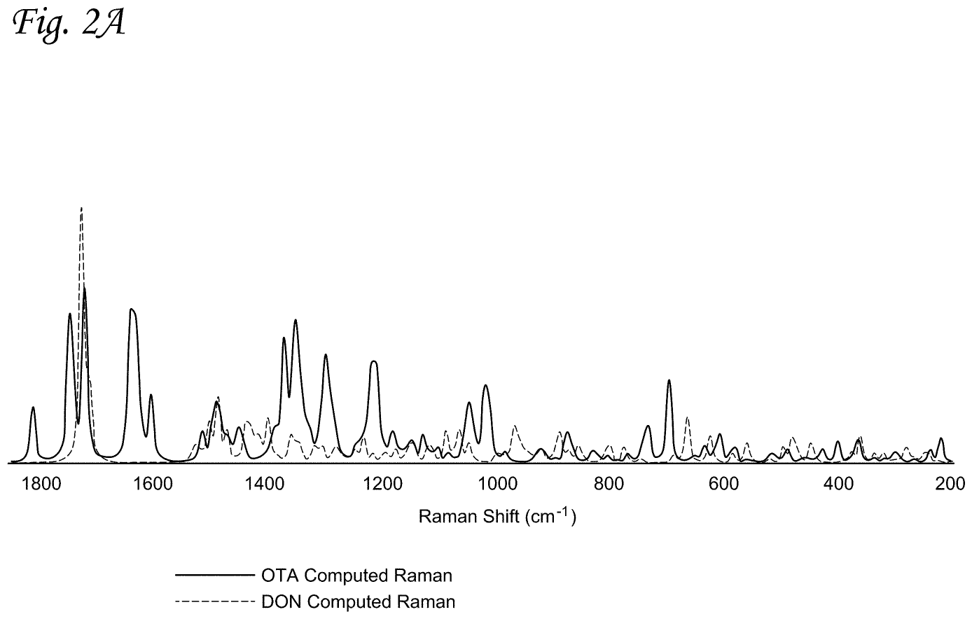

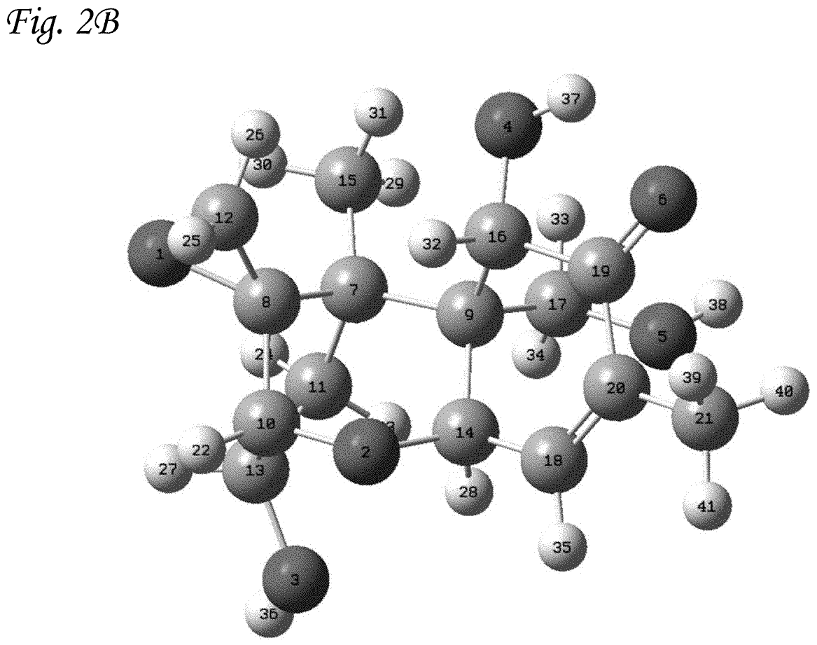

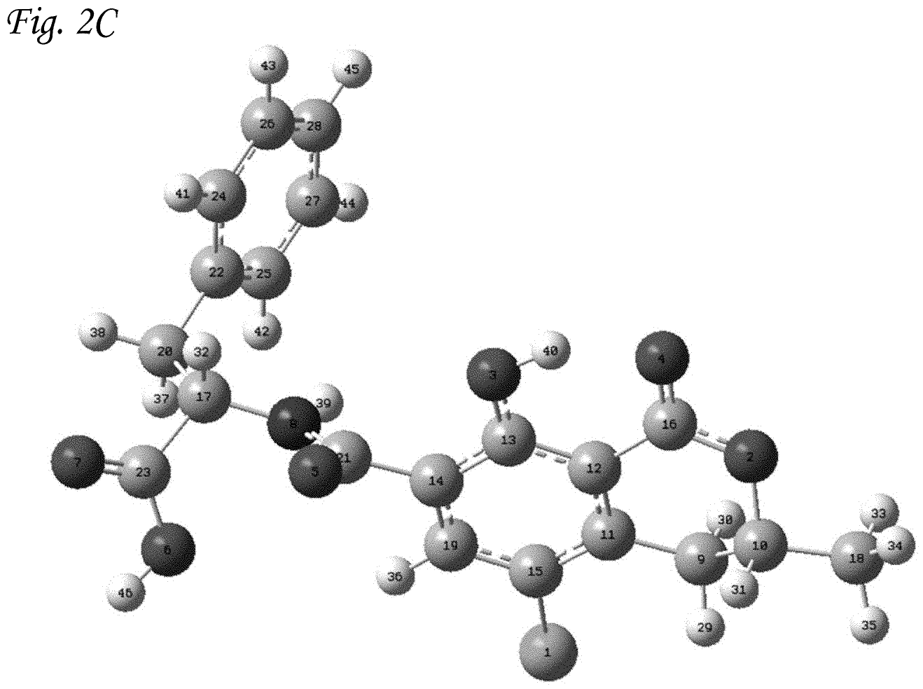

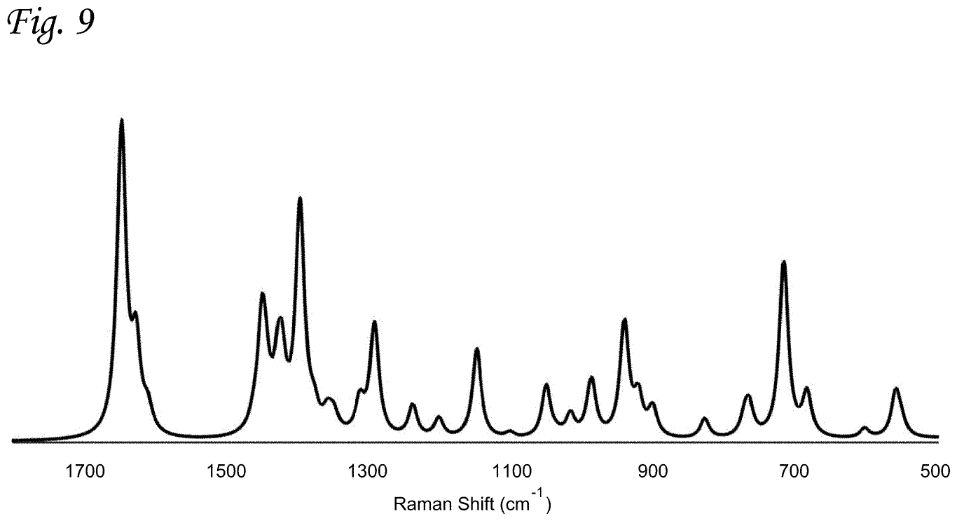

Computational modeling of the molecule was completed here to fully assign all possible peaks in the experimental spectrum for DON. To determine vibrational band assignments for OTA itself, DFT calculations were performed for both mycotoxins, and their vibrational band assignments were labeled accordingly. The Raman spectrum of DON was computed following the same basis set as previously reported and agreed well with published calculations. As can be seen in FIG. 2, the spectra of the two mycotoxins DON and OTA had some overlap. There were a sufficient number of stand-alone peaks for each molecule indicating the potential for successful multiplexing. Previous computational work has labeled the vibrational modes of the AEMA monomer and the CTA anchoring group that make up the affinity agent. (Szlag, V. M. et al Anal. Chem. 2018.) The computed Raman spectrum for the AEMA monomer can be seen in FIG. 9.

### Example 2

SERS Detection of Deoxynivalenol with pAEMA₂₉.

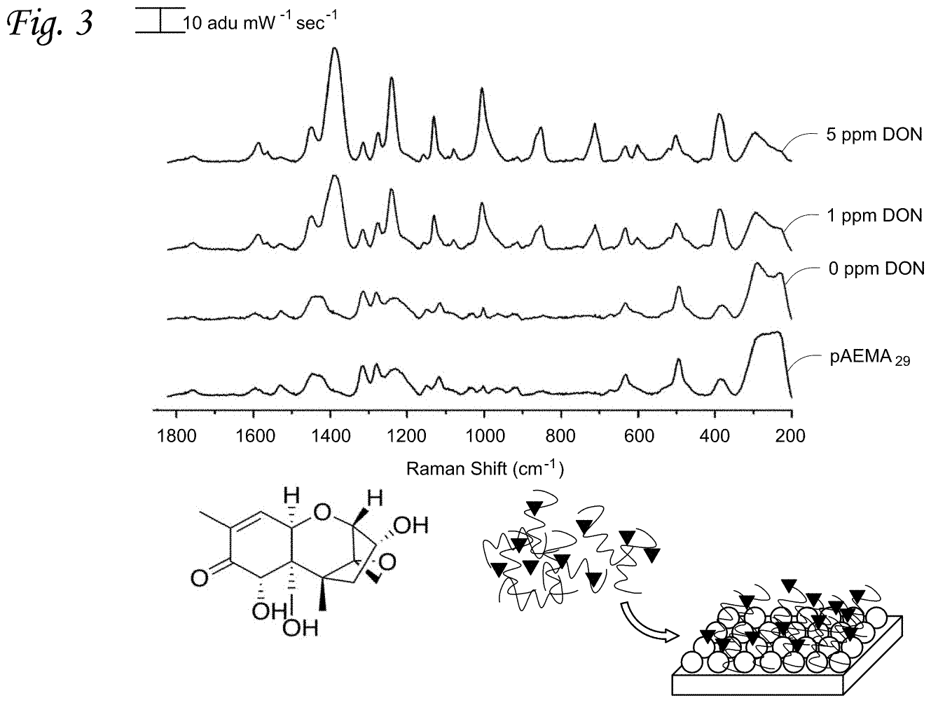

In contrast to traditional affinity agents, which have been anchored to the sensing substrate and then flowing or attaching the target, DON and pAEMAa₂₉ were complexed in solution, through what is hypothesized to be hydrogen bonding, and then this complex was anchored to the FON substrate. Comparing the anchored complex at various concentrations of DON to DON-free polymer and the 0 ppm condition (just the solution conditions the toxin is diluted in) in FIG. 3 visibly revealed distinct spectral features, changes in peak intensity, and peak shifts. DON's vibrational signature was apparent at 1 ppm, the concentration at which the toxin is regulated. Additionally, FONs were incubated in varying DON solutions, without the polymer affinity agent, and spectral features were only seen at DON concentrations significantly higher than the concentration relevant for real-world sensing By comparing vibrational modes in the DON computational spectrum, the monomer/CTA spectrum, and the experimental spectra, binding between the small molecule and the polymer were confirmed. A comparison of DON and monomer/CTA DFT vibrational modes to experimental vibrational modes can be seen in Table 2.

TABLE 2
Table of select vibrational modes comparing computational
DON and monomer/CTA to experimental spectra.

| | DON | Monomer/CTA | Monomer/CTA |
|---|---|---|---|
| | Experimental | Calculated | Experimental |
| DON DFT Calculated Raman Shift (cm⁻¹ | Raman Shift | Raman Shift | Raman Shift |
| shift) | (cm⁻¹ shift) | (cm⁻¹ shift) | (cm⁻¹ shift) |
| 1530: scissoring of H's on C12, twisting of | 1590 and 1566 | 1610: monomer bending | 1607 |
| H's on C15 | shoulder | of N2 H's | |
| 1450: Asymmetric out of plane rocking of | 1455 | 1423 and 1428: | 1433 |
| H's on C17 and wagging of H38 on O5, | | O1—C4—N1 | |
| wagging of H37 on O4, slight wagging of | | symmetric stretch, N1—C5 | |
| H's on C11 | | stretch, rocking of H | |
| | | on N1 and asymmetric | |
| | | bending of C1 H's | |
| | | respectively | |
| 1384: Rocking of H37 on O4, symmetric | 1388 | | |
| angle bending of C18—C14 displacing H35 | | | |
| and H28, rocking of Hs on C21, ring | | | |
| rocking of cyclohexene | | | |
| 1245: Strong stretching of H22 on C10, | 1241 | 1238: twisting/wagging of | 1234 |
| symmetric stretching of H27 on C13 and | | H's on N1, C5, C6, and | |
| O3 on C13, strong angle wagging of H38 | | N2 | |
| on O5 and H33 on C17 | | | |
| 853: Ring breathing C10, H's on C11 in | 852 | | |
| plane stretch, H32 rocking, H35 bend | | | |

### Example 3

SERS Detection of Ochratoxin A with pAEMA₂₉.

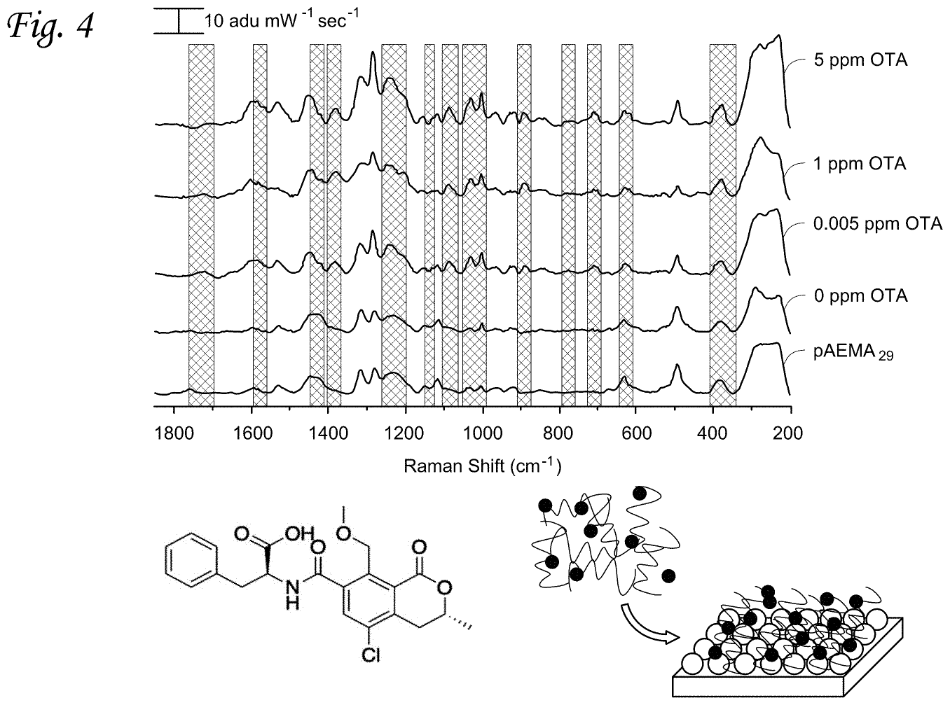

Following the same protocol as with DON, FONs were incubated in an OTA and pAEMA₂₉ mixture at varying concentrations, and the captured spectra can be seen in FIG. 4. Distinct and visible spectral changes are observed at concentrations as low as the OTA regulatory limit (0.005 ppm/5 ppb). A comprehensive table comparing OTA and monomer/CTA DFT vibrational modes to the experimental modes can be seen in Table 3. FONs were incubated in varying OTA concentrations, without polymer affinity agent and it was not possible to detect OTA at relevant concentrations.

TABLE 3
Table of select vibrational modes comparing computational
OTA and monomer/CTA to experimental spectra.

| | OTA | Monomer/CTA | Monomer/CTA |
|---|---|---|---|
| | Experimental | Calculated | Experimental |
| | Raman Shift | Raman Shift | Raman Shift |
| OTA DFT Calculated Raman Shift (cm⁻¹) | (cm⁻¹) | (cm⁻¹) | (cm⁻¹) |
| 1378: Strong H32 wagging on C17, asymmetric | 1384 | | |
| wagging of Hs on C20, slight twisting of | | | |
| benzene ring Hs (H42, H44, etc.) | | | |
| 1082: Strong C9 H's stretch causing a strong | 1089 | | |
| inner ring distortion on C9, C10, C11, wagging | | | |
| of H39, asymmetric stretch of benzene ring at | | | |
| C11, wagging of H40 | | | |
| 898: In plane wagging of H's on C18, angle | 891 | | |
| breath of C10 and C18, C9 stretching | | | |
| 714: Asymmetric out of plane breathing of | 713 | | |
| benzene ring, strong displacement of H41, H42, | | | |
| and H45 | | | |
| 654: Outward bond angle stretch of C23═O7 | 676 | 654: S3—C5 | 650 |
| and O6 and H46, twisting of H's on C20, ring | | stretch | |
| distortion at C11 and twisting of H29, ring twist | | (trithiocarbonate) | |
| displacing H43 | | | |

### Example 4

Multiplex SERS Detection of Deoxynivalenol and Ochratoxin a with pAEMA₂₉

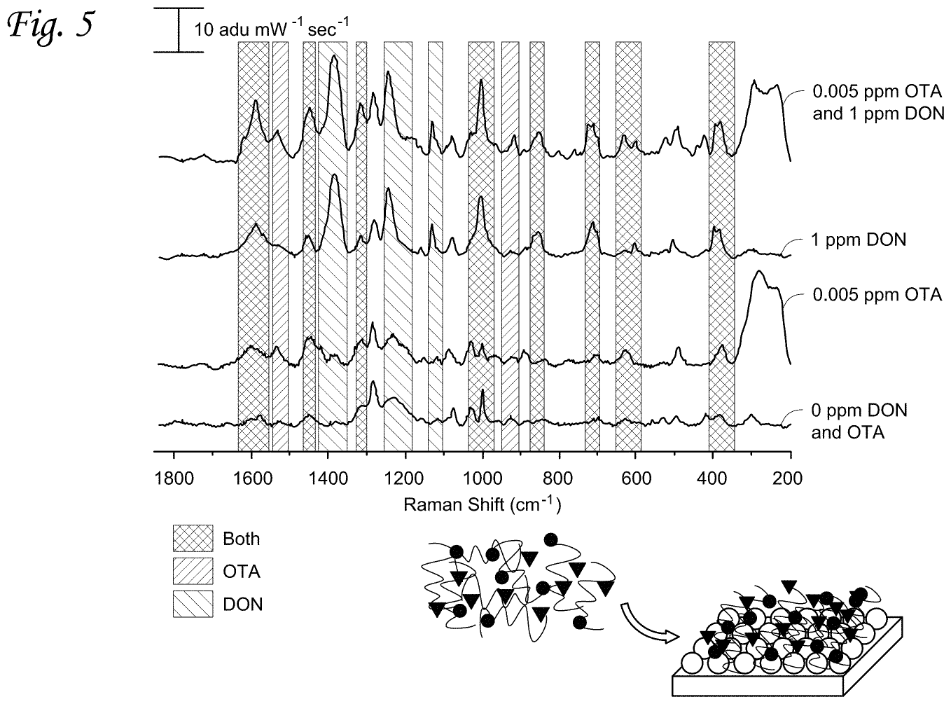

In an effort to detect both toxins simultaneously, both DON and OTA were placed in solution with pAEMA₂₉ to give both the opportunity to complex with the polymer affinity agent via the hypothesized hydrogen bonding/association. The multiplexed spectra, seen in FIG. 5 display vibrational band character from both mycotoxins. While there was significant overlap between the two spectra of DON and OTA, as predicted by the computational spectra in FIG. 2, there were still stand-alone vibrational modes that corresponded to each toxin individually, implying that each toxin can be distinguished from the other.

The association between mycotoxin and our polymer system was hypothesized to be through hydrogen bonding interactions, while other mycotoxin and affinity agent sensing systems have relied on much more specific interactions. Previous computational modeling to screen various monomers to bind to two different mycotoxins for chromatography applications has revealed a strong binding energy (−41.94 kcal/mol) between OTA and a monomer structure with amine groups. (Piletska, E. et al., Development of the Custom Polymeric Materials Specific for Aflatoxin B1 and Ochratoxin A for Application with the ToxiQuant T1 Sensor Tool, J Chromatogr A 2010, 1217 (16), 2543-2547.) The monomer modeled in that work was not easily amenable to controlled polymerizations. In this example, the same interactions were exploited with a sensing system of the present disclosure due to the amine groups on the polymer and the moieties on both OTA and DON that readily interact with amines through hydrogen bonding. By monitoring the stand-alone, unique peaks to each mycotoxin in the multiplex spectra, the types of interactions occurring were confirmed while sensing the two small molecules simultaneously.

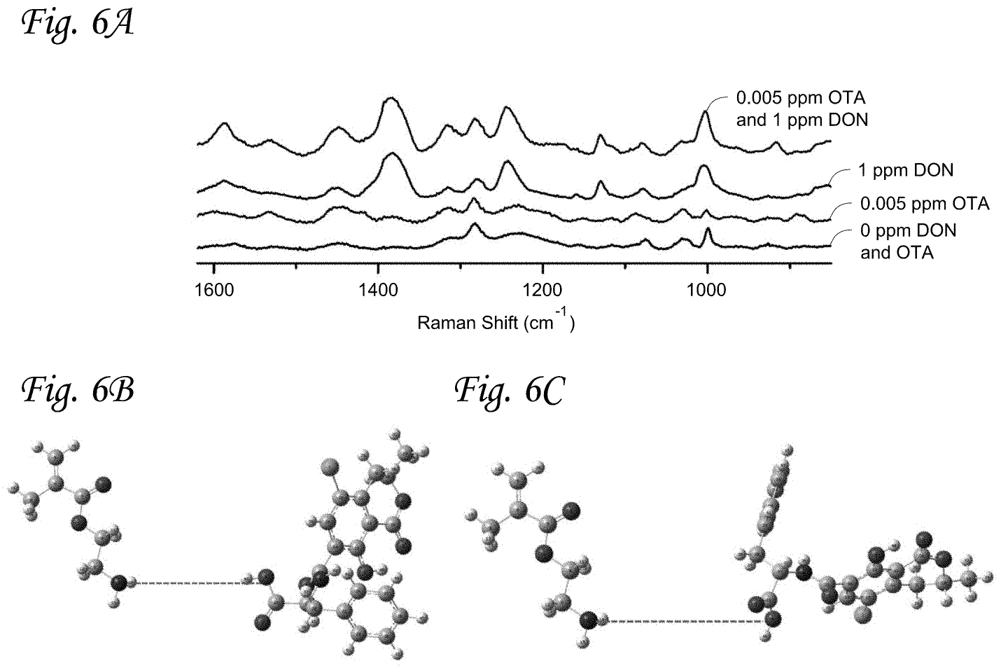

The carboxyl group on the phenylalanine moiety of OTA can form hydrogen bonds between amino groups on a monomer and the carboxyl group of the phenylalanine moiety and electrostatically interact with the amino group on the primary amine monomers. (Piletska, E. et al., J. Chromatogr A 2010.) In the multiplex spectra, the 1535 cm⁻¹ shift, unique to OTA, was referenced to the 1527 cm⁻¹ shift in the computational spectrum. When monitoring this computational mode in real time, the stretch was attributed to strong vibrations on the benzyl ring of the phenylalanine moiety of OTA. This may indicate hydrogen bonding at the carboxyl of the OTA phenylalanine. The second stand-alone peak in the multiplex spectra, at the 916 cm′ shift, was referenced to the 917 cm⁻¹ shift in the computational spectrum due to strong asymmetric stretching of the tertiary carbons attached to the carboxyl group previously mentioned. This further confirmed hydrogen bonding between OTA and the linear polymer affinity agent. An enlarged image of the multiplex spectra and hypothesized binding can be seen in FIG. 6.

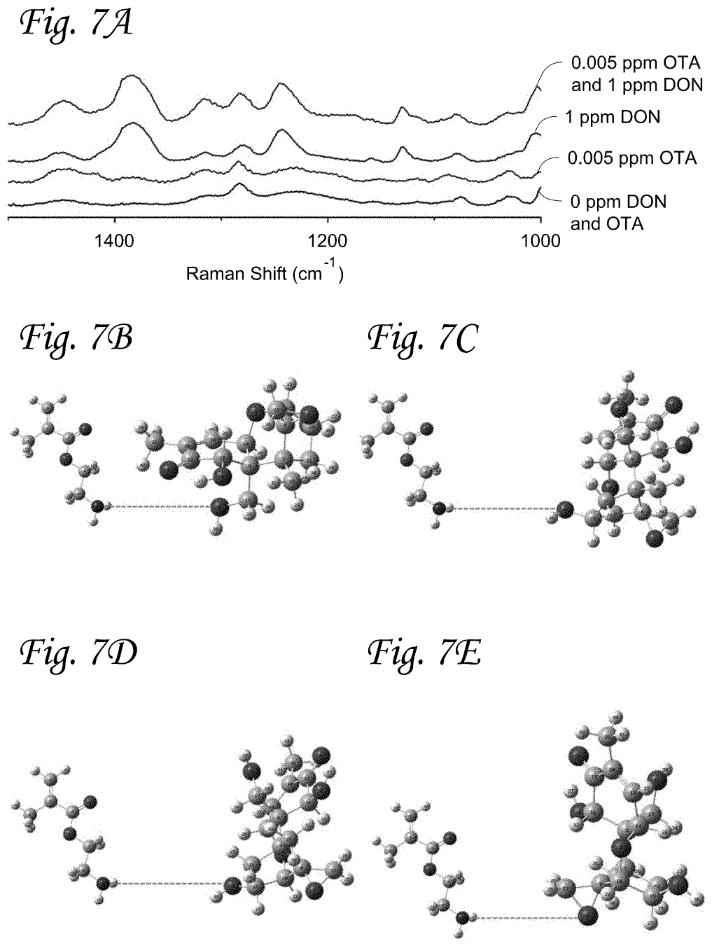

Following the same hypotheses for OTA and pAEMA₂₉, observing the unique DON peaks in the multiplex spectra, molecular details about DON interactions with the polymer affinity agent were examined. The peak at 1455 cm⁻¹ shift in the multiplex spectra was referenced to 1450 cm⁻¹ shift from the computational spectrum, strong asymmetric rocking of the hydrogens on a tertiary carbon bound to a hydroxyl group, indicating hydrogen bonding at the hydroxyl. At 1234 cm⁻¹ shift, the polymer and blank spectra have a broadened peak that shifts and sharpens into a peak at 1241 cm⁻¹ shift that were referenced to the 1245 cm⁻¹ shift in the computational spectrum of DON. This vibrational mode was attributed to both strong stretching of the hydrogens on the bicyclic ring near the hydroxyl (O3) and symmetric stretching of the hydrogens in the out of plane hydroxyl group (O5) as labeled in FIG. 6. Lastly, the sharp 1130 cm⁻¹ shift of DON in the multiplex spectra were referenced to strong stretches at 1142 and 1146 cm⁻¹ shifts in the computational spectrum. These strong vibrations are hypothesized to be due to hydrogen stretches on the bicyclic ring near the hydroxyl (O3), with movement in the epoxide ring and symmetric stretching in the epoxide ring hydrogens with symmetric stretching of the hydrogens in the out-of-plane hydroxyl group (O5), respectively as labeled in FIG. 7.

Thus, various embodiments of linear polymer affinity agent substrate for surface-enhanced Raman spectroscopy are disclosed. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern.

References explicitly incorporated herein include, but are not limited to, the following:
(1) Szlag, V. M. et al. Molecular Affinity Agents for Intrinsic Surface-Enhanced Raman Scattering (SERS) Sensors. ACS Appl. Mater. Interfaces 2018, 10 (38), 31825-31844.
(2) Szlag, V. M. et al. Isothermal Titration calorimetry for the Screening of Aflatoxin B1 Surface-Enhanced Raman Scattering Sensor Affinity Agents. Anal. Chem. 2018, 90 (22), 13409-13418.
(3) Szlag, V. M. et al. Optimizing Linear Polymer Affinity Agent Properties for Surface-Enhanced Raman Scattering Detection of Aflatoxin B1. Mol. Syst. Des. Eng. 2019.

What is claimed is:

1. A method of using a sensor, the method comprising:
mixing a linear polymer affinity agent in a sample solution;
subjecting a metal substrate to the sample solution to attach the linear polymer affinity agent to the metal substrate;
generating, via Raman Spectroscopy, spectral data representing the linear polymer affinity agent attached to the metal substrate;
determining whether two or more analytes are present in the sample solution at respective minimum threshold concentrations based on the spectral data.

2. The method of claim 1, wherein the linear polymer affinity agent comprises methacrylamide.

3. The method of claim 1, wherein the linear polymer affinity agent is synthesized via polymerization of N-(2-aminoethyl) methacrylamide hydrochloride.

4. The method of claim 1, wherein determining whether two or more analytes are present comprises determining whether two or more mycotoxins are present.

5. The method of claim 1, wherein determining whether two or more analytes are present comprises determining whether deoxynivalenol, ochratoxin A, or both are present.

6. The method of claim 1, wherein determining whether two or more analytes are present comprises detecting one or more hydrogen bonds between the linear polymer affinity agent and at least one of the analytes.

7. The method of claim 1, wherein determining whether two or more analytes are present comprises identifying a peak in the spectral data not associated with the linear polymer affinity agent or the two or more analytes.

8. The method of claim 1, wherein determining whether two or more analytes are present comprises identifying bonds with a different functional group of each toxin bonding with the linear polymer affinity agent.

9. The method of claim 1, wherein determining whether two or more analytes are present comprises identifying a peak in a range from 300 to 1700 cm⁻¹ as corresponding to ochratoxin A.

10. The method of claim 1, wherein determining whether two or more analytes are present comprises identifying a peak in a range from 300 to 1800 cm⁻¹ as corresponding to deoxynivalenol.

11. A sensor comprising:
a metal substrate comprising a plasmonic metal; and
at least one linear polymer affinity agent synthesized via polymerization of N-(2-aminoethyl) methacrylamide hydrochloride (pAEMA) and attached to the metal substrate to bind to a toxin.

12. The sensor of claim 11, wherein the polymer affinity agent comprises pAEMA₂₉.

13. The sensor of claim 11, wherein the metal substrate comprises at least one of gold, copper, and silver.

14. The sensor of claim 11, wherein the metal substrate comprises one of a film of gold over silica nanosphere matrix and a colloidal gold substrate.

15. The sensor of claim 11, wherein the at least one linear polymer affinity agent is configured to bind to at least two mycotoxins.

16. The sensor of claim 11, wherein the at least one linear polymer affinity agent is configured to bind deoxynivalenol, ochratoxin A, or both.

17. A method of calibrating a sensor, the method comprising:
subjecting a metal substrate to a calibrating solution comprising at least one linear polymer affinity agent and at least two analytes at respective known concentrations;
generating, via Raman Spectroscopy, spectral data representing the at least one linear polymer affinity agent being bound to the at least two analytes and being attached to the metal substrate;
generating calibration data based on the spectral data to detect the at least two analytes at respective minimum threshold concentrations, wherein the calibration data includes identification of different peaks associated with each toxin.

18. The method of claim 17, wherein the at least two analytes comprise two mycotoxins.

19. The method of claim 17, wherein the at least two analytes comprise deoxynivalenol, ochratoxin A, or both.
